SEQUENCE LISTING (33 sequences)

SEQ ID NO: 1 - DNA, 9 bp, Artificial Sequence
  Synthetic Construct
ttggggtat

SEQ ID NO: 2 - DNA, 9 bp, Artificial Sequence
  Synthetic Construct
ccgaagggt

SEQ ID NO: 3 - DNA, 9 bp, Artificial Sequence
  Synthetic Construct
ccgagatac

SEQ ID NO: 4 - DNA, 9 bp, Artificial Sequence
  Synthetic Construct
ccgaaggat

SEQ ID NO: 5 - DNA, 9 bp, Artificial Sequence
  Synthetic Construct
ctgggggat

SEQ ID NO: 6 - DNA, 9 bp, Artificial Sequence
  Synthetic Construct
ttgggatac

SEQ ID NO: 7 - DNA, 9 bp, Artificial Sequence
  Synthetic Construct
ccagagggc

SEQ ID NO: 8 - DNA, 9 bp, Artificial Sequence
  Synthetic Construct
ccagagggt

SEQ ID NO: 9 - DNA, 9 bp, Artificial Sequence
  Synthetic Construct
ctggggtat

SEQ ID NO: 10 - DNA, 9 bp, Artificial Sequence
  Synthetic Construct
ccgggggac

SEQ ID NO: 11 - DNA, 9 bp, Artificial Sequence
  Synthetic Construct
ttggggtac

SEQ ID NO: 12 - DNA, 8 bp, Artificial Sequence
  Synthetic Construct
aagggtgc

SEQ ID NO: 13 - DNA, 8 bp, Artificial Sequence
  Synthetic Construct
aaggagat

SEQ ID NO: 14 - DNA, 8 bp, Artificial Sequence
  Synthetic Construct
cggaagat

SEQ ID NO: 15 - DNA, 8 bp, Artificial Sequence
  Synthetic Construct
cgggagat

SEQ ID NO: 16 - DNA, 8 bp, Artificial Sequence
  Synthetic Construct
aggaggat

SEQ ID NO: 17 - DNA, 8 bp, Artificial Sequence
  Synthetic Construct
cggaggat

SEQ ID NO: 18 - DNA, 8 bp, Artificial Sequence
  Synthetic Construct
agggagat

SEQ ID NO: 19 - DNA, 8 bp, Artificial Sequence
  Synthetic Construct
aagaggat

SEQ ID NO: 20 - DNA, 8 bp, Artificial Sequence
  Synthetic Construct
cggggtgc

SEQ ID NO: 21 - DNA, 8 bp, Artificial Sequence
  Synthetic Construct
agagagat

SEQ ID NO: 22 - DNA, 8 bp, Artificial Sequence
  Synthetic Construct
aggggtgc

SEQ ID NO: 23 - DNA, 8 bp, Artificial Sequence
  Synthetic Construct
aggggtat

SEQ ID NO: 24 - DNA, 8 bp, Artificial Sequence
  Synthetic Construct
aagagggt

SEQ ID NO: 25 - DNA, 8 bp, Artificial Sequence
  Synthetic Construct
cggggggc

SEQ ID NO: 26 - DNA, 7 bp, Artificial Sequence
  Synthetic Construct
ccagcac

SEQ ID NO: 27 - DNA, 7 bp, Artificial Sequence
  Synthetic Construct
tcagtac

SEQ ID NO: 28 - DNA, 7 bp, Artificial Sequence
  Synthetic Construct
ttgatgc

SEQ ID NO: 29 - DNA, 7 bp, Artificial Sequence
  Synthetic Construct
ttaacac

SEQ ID NO: 30 - DNA, 7 bp, Artificial Sequence
  Synthetic Construct
tcagtaa

SEQ ID NO: 31 - DNA, 7 bp, Artificial Sequence
  Synthetic Construct
ttagcac

SEQ ID NO: 32 - DNA, 7 bp, Artificial Sequence
  Synthetic Construct
ccagtac

SEQ ID NO: 33 - DNA, 7 bp, Artificial Sequence
  Synthetic Construct
ttaatac

(19) United States
(12) Patent Application Publication
(10) Pub. No.: US 2013/0040838 A1
(43) Pub. Date: Feb. 14, 2013

(54) METHODS FOR IDENTIFYING THE PRESENCE OF A BICUSPID AORTIC VALVE

(71) Applicants: Gordon Huggins, Bedford, MA (US); Eric Wooten, Boston, MA (US); Michael Mendelsohn, Boston, MA (US)

(72) Inventors: Gordon Huggins, Bedford, MA (US); Eric Wooten, Boston, MA (US); Michael Mendelsohn, Boston, MA (US)

(73) Assignee: Tufts Medical Center ,Inc., Boston, MA (US)

(21) Appl. No.: 13/520,095

(22) PCT Filed: Aug. 20, 2010
(86) PCT No.: PCT/US2010/046148

§ 371 Date: Oct. 23, 2012

Related U.S. Application Data
(60) Provisional application No. 61/293,886, filed on Jan. 11, 2010.

(51) Int. Cl. C40B 30/04; C07H 21/04; C40B 40/06; C12Q 1/68

(52) U.S. Cl. 506/9; 435/6.11; 536/24.31; 536/24.33; 506/16

(57) ABSTRACT

The present invention features a method for identifying a subject with a bicuspid aortic valve (BAV) by detecting one or more single nucleotide polymorphisms (SNPs) present in one or more BAV-associated chromosomal regions (e.g., chromosomal regions containing the AXIN1-PDIA2, ENG, BAT2/3, or ZNF385D gene(s)).

### STATEMENT AS TO FEDERALLY SPONSORED RESEARCH

This invention was made with government support under grant number 0826005D from the American Heart Association. The government has certain rights in the invention.

### BACKGROUND OF THE INVENTION

In general, the invention relates to a method for identifying a bicuspid aortic valve (BAV) in a subject.

The aortic valve, as formed during embryonic heart development, is comprised of three cusps divided by three commissures. Cusp fusion, or the failure of the cusps to separate during heart development, can produce a valve with two cusps (i.e., bicuspid) or one cusp (i.e., unicuspid). In a bicuspid valve, the two conjoined cusps form a larger cusp that operates with the remaining normal cusp to perform the function of the valve.

Despite a high heritability, challenges remain in determining the genetic cause of a BAV. First, few multigenerational pedigrees contain a BAV as an isolated trait to enable gene identification by traditional approaches. Second, large, well-phenotyped BAV cohorts are not readily available due to the relative rarity of a BAV in the general population. Third, it can he difficult to determine non-invasively whether a thickened and calcified aortic valve is bicuspid or tricuspid in adults. Direct, surgical inspection is often required to determine whether a BAV is present, which limits subject recruitment. Finally, the phenotypic diversity observed within a BAV implies an assortment of developmental pathways ultimately converging around a single, uniquely identifiable phenotypic outcome: aortic valve formation and valve disease. Thus, understanding the genetic underpinnings of BAV formation may help to identify diagnostic markers of bicuspid aortic valves.

There exists a need in the art for additional methods for identifying subjects with a BAV.

### SUMMARY OF THE INVENTION

The present invention features a method for identifying a subject with a bicuspid aortic valve (BAV) by detecting one or more single nucleotide polymorphisms (SNPs) present in one or more BAV-associated chromosomal regions (e.g., chromosomal regions containing the AXIN1-PDIA2, ENG, BAT2/3, or ZNF385D gene(s)).

In one aspect, the invention features a method for identifying a subject (e.g., a human) with a BAV by detecting in a biological sample obtained from a subject at least one SNP in one or more BAV-associated chromosomal regions selected from AXIN1-PDIA2, ENG, BAT2/3, and/or ZNF385D, wherein the presence of at least one SNP identifies the subject as having a BAV.

In certain embodiments, the subject has a personal or family history of heart defects or heart disease.

In other embodiments, the biological sample is obtained from heart tissue or peripheral blood and includes nucleic acid (e.g., DNA, genomic DNA, RNA, cDNA, hnRNA, or mRNA). The nucleic acid may be extracted and purified for further analysis.

In other embodiments, the detection step of the first aspect may include one or more of oligonucleotide microarray analysis, allele-specific hybridization, allele-specific polymerase chain reaction (PCR), 5′ nuclease digestion, molecular beacon assay, oligonucleotide ligation assay, size analysis, or nucleic acid sequencing.

In a second aspect, the invention features a kit that includes an assay for detecting at least one SNP in one or more BAV-associated chromosomal regions selected from AXIN1-PDIA2, ENG, BAT2/3, and/or ZNF385D, wherein the presence of at least one SNP identifies a subject as having a BAV. In certain embodiments, the kit additionally includes instructions for correlating assay results with the presence of BAV in a subject.

In a final aspect, the invention features a microarray that includes oligonucleotide probes capable of hybridizing under stringent conditions to one or more nucleic acid molecules having at least one SNP in one or more BAV-associated chromosomal regions selected from AXIN1-PDIA2, ENG, BAT2/3, and/or ZNF385D.

In any of the aspects of the present invention, the SNP may be one or more of rs2685127, rs419949, rs12925669, rs214247, rs1981492, rs2301522, rs7359414, rs3916990, rs9921222, rs400037, rs4451422, rs10819309, rs3739817, rs11792480, rs10121110, rs11789185, rs4837192, rs10987759, rs2261033, rs3132453, rs388647, rs800621, and/or rs711735.

By “allele” or “allelic variant” is meant a polynucleotide sequence variant of a gene of interest. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other by a single nucleotide or several nucleotides and such differences can include substitutions, deletions, and insertions of nucleotides. An allele of a gene can also be a form of a gene containing a mutation.

By an “amplified” nucleic acid is meant a nucleic acid with an increased number of copies. The amplification may be across several orders of magnitude, generating thousands to millions of copies of a particular nucleic acid sequence. Methods for amplification include, e.g., polymerase chain reaction (PCR), ligation amplification (or ligase chain reaction (LCR)), or any other amplification method known to one of skill in the art.

By “AXIN1-PDIA2 chromosomal region” is meant a region on chromosome 16p13.3 that includes the AXis INhibitor 1 (AXIN1) and Protein Disulfide Isomerase family A, member 2 (PDIA2) genes. By “AXIN1” is meant a polynucleotide having the genomic nucleic acid sequence of NCBI Reference Sequence: NG_{—}012267.1 and the mRNA sequence of NCBI Reference Sequence: NM_{—}003502.2 (isoform a) or NM_{—}181050.1 (isoform b) or a polypeptide having the amino acid sequence of NCBI Reference Sequence: NP_{—}003493.1 (isoform a) or NP 851393.1 (isoform b). By “PDIA2” is meant a polynucleotide having the genomic nucleic acid sequence of NCBI Reference Sequence: NC_{—}000016.9 and the mRNA sequence of NCBI Reference Sequence: NM_{—}006849.2 or a polypeptide having the amino acid sequence of NCBI Reference Sequence: NP_{—}006840.2.

By “BAT2/3 chromosomal region” is meant a region on chromosome 6 that includes the HLA-B Associated Transcript 2 (BAT2) and 3 (BAT3) genes. By “BAT2” is meant a polynucleotide having the genomic nucleic acid sequence of NCBI Reference Sequence: NC_{—}000006.11 and the mRNA sequence of NCBI Reference Sequence: NM_{—}080686.2 or a polypeptide having the amino acid sequence of NCBI Reference Sequence: NP_{—}542417.2. By “BAT3” is meant a polynucleotide having the genomic nucleic acid sequence of NCBI Reference Sequence: NC_{—}000006.11 and the mRNA sequence of NCBI Reference Sequence: NM_{—}004639.3 (isoform a) or NM_{—}001098534.1 (isoform b) or a polypeptide having the amino acid sequence of NCBI Reference Sequence: NP_{—}004630.3 (isoform a) or NP_{—}001092004.1 (isoform b).

By “bicuspid aortic valve” or “BAV” is meant a defect of the aortic valve that results in the formation of two leaflets or cusps instead of the normal three (i.e., a tricuspid valve). In addition to the methods described herein, diagnosing the presence of a bicuspid aortic valve can be made by identifying a heart murmur at the right second intercostal space; identifying differences in blood pressure between upper and lower extremities; and imaging via echocardiography (ECG) and magnetic resonance imaging (MRI).

By “chromosomal region containing a gene” is meant a portion of a chromosome that includes one or more genes. In certain embodiments, the chromosomal region can include nucleotide sequences adjacent to both ends of the gene(s). Exemplary chromosomal regions of the present invention (e.g., BAV-associated chromosomal regions) include, without limitation, chromosomal regions containing the AXIN1-PDIA2, ENG, BAT2/3, and/or ZNF385D gene(s).

By “ENG chromosomal region” is meant a region on chromosome 9 that includes the endoglin (ENG) gene. By “ENG” is meant a polynucleotide having the genomic nucleic acid sequence of NCBI Reference Sequence: NG_{—}009551.1 and the mRNA sequence of NCBI Reference Sequence: NM_{—}001114753.1 (isoform 1) or NM_{—}000118.2 (isoform 2) or a polypeptide having the amino acid sequence of NCBI Reference Sequence: NP_{—}001108225.1 (isoform 1) or NP_{—}000109.1 (isoform 2).

By “extracted” or “isolated” is meant a procedure to collect a nucleic acid from a sample. Methods of preparing nucleic acid extracts are well known in the art and can be readily adapted to obtain a sample that is compatible with the system utilized. Automated sample preparation systems for extracting nucleic acids from a test sample are commercially available, and examples include Qiagen's BioRobot 9600, Applied Biosystems' PRISM 6700, and Roche Molecular Systems' COBAS AmpliPrep System.

By “genotype” is meant the alleles of a gene contained in an individual or a sample. In the context of this invention, no distinction is made between the genotype of an individual and the genotype of a sample originating from the individual.

By “microarray” is meant an arrayed series of microscopic spots of oligonucleotides, each spot containing a specific nucleic acid sequence (e.g., a probe sequence). The specific nucleic acid sequences can be a short section of a gene or other nucleic acid element that is used to hybridize a nucleic acid sample (e.g., a target sample) under high-stringency conditions. In one embodiment, the microarray includes polynucleotides representative of BAV-associated chromosomal regions (e.g., chromosomal regions containing AXIN1-PDIA2, ENG, BAT2/3, and/or ZNF385D genes and SNPs thereof).

By “polymorphic” or “polymorphism” is meant the occurrence of two or more genetically determined alternative sequences of a gene in a population. The polymorphic region or polymorphic site refers to a region of the polynucleotide where the nucleotide difference that distinguishes the variants occurs. Typically, the first identified allelic form is arbitrarily designated as the reference form and other allelic forms are designated as alternative or variant alleles. The allelic form occurring most frequently in a selected population is sometimes referred to as the wild-type form.

By “polynucleotide” or “nucleic acid” is meant any polyribonucleotide or polydeoxyribonucleotidc, which may be unmodified RNA or DNA or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, hybrid molecules comprising DNA and RNA that may be single-stranded, or, more typically, double-stranded or a mixture of single- and double-stranded regions. In addition, polynucleotide refers to triple-stranded regions comprising RNA or DNA or both RNA and DNA. Nucleic acids may include, without limitation, mRNA, tRNA, rRNA, tmRNA, miRNA, siRNA, piRNA, aRNA, snRNA, snoRNA, shRNA, cDNA, msDNA, and mtDNA.

The term polynucleotide also includes DNAs or RNAs containing one or more modified bases and DNAs or RNAs with backbones modified for stability or for other reasons. Modified bases include, for example, tritylated bases and unusual bases such as inosine. A variety of modifications can be made to DNA and RNA; thus, polynucleotide embraces chemically, enzymatically, or metabolically modified forms of polynucleotides as typically found in nature, as well as the chemical forms of DNA and RNA characteristic of viruses and cells. Polynucleotide also embraces short nucleic acid chains, often referred to as oligonucleotides.

By “sample” is meant solid and fluid samples. By “biological sample” is meant cells (e.g., cardiomyocytes), protein or membrane extracts of cells, or blood (e.g., peripheral blood), or biological fluids including, e.g., ascites fluid or brain fluid (e.g., cerebrospinal fluid (CSF)). Examples of solid biological samples include samples taken from feces, the rectum, central nervous system, bone, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, and the thymus. Examples of biological fluid samples include samples taken from the blood, serum, CSF, semen, prostate fluid, seminal fluid, urine, saliva, sputum, mucus, hone marrow, lymph, and tears. Samples may be obtained by standard methods including, e.g., venous puncture and surgical biopsy. In certain embodiments, the biological sample is a heart, breast, lung, colon, or prostate tissue sample obtained by needle biopsy.

By “single-nucleotide polymorphism” or “SNP” is meant a polynucleotide that differs from another polynucleotide by a single nucleotide exchange. For example, exchanging one adenine (A) for one cytosine (C), guanine (G), or thymine (T) in the entire sequence of a polynucleotide constitutes a SNP. Single-nucleotide polymorphisms may occur in coding regions (e.g., protein-coding regions) of genes, non-coding regions of genes, or in the intergenic regions between genes. SNPs within a coding sequence will not necessarily change the amino acid sequence of the protein that is produced due to degeneracy of the genetic code. SNPs that are not in coding regions may still have consequences for gene splicing, transcription factor binding, or the sequence of non-coding RNA.

By “stringent conditions” is meant a condition under which a specific nucleic acid hybrid is formed and non-specific nucleic acid hybrid is not formed. Stringency can be modified by, for example, modifying temperature and/or salt concentration. Detection of specific nucleic acid sequences with moderate or high similarity to, for example, an oligonucleotide probe depends on the stringency of the hybridization conditions. High stringency, such as high hybridization temperature and low salt in hybridization buffers, permits only hybridization between nucleic acid sequences that are highly similar, whereas low stringency, such as lower temperature and high salt, allows hybridization when the sequences are less similar.

By “subject” is meant any animal, e.g., a mammal (e.g., a human). Other animals that can be diagnosed using the methods of the invention include, e.g., horses, dogs, cats, pigs, goats, rabbits, hamsters, monkeys, guinea pigs, rats, mice, lizards, snakes, sheep, cattle, fish, and birds.

By “target sequence” or “target region” is meant a region of a nucleic acid that is to be analyzed and comprises the polymorphic site of interest.

By “ZNF385D chromosomal region” is meant a region on chromosome 3 that includes the zinc finger protein 385D (ZNF385D). By “ZNF385D” is meant a polynucleotide having the genomic nucleic acid sequence of NCBI Reference Sequence: NC_{—}000003.11 and the mRNA sequence of NCBI Reference Sequence: NM_{—}024697.2 or a polypeptide having the amino acid sequence of NCBI Reference Sequence: NP_{—}078973.1.

Other features and advantages of the invention will be apparent from the detailed description and from the claims.

### BRIEF DESCRIPTION OF THE DRAWINGS

FIG. 1 is a table showing the frequency of major ontology classes of genes differentially expressed in the aorta of subjects with BAV. These classes are present in the combined prioritized gene lists returned by CANDID and STRING. Frequency counts and fractional percentage were determined relative to the total number of observations.

FIG. 2 is a schematic representation showing the methodology for identifying SNPs associated with BAV. The 311,399 SNPs present on the Illumina CNV370 (representing about 15,000 well-annotated genes) were prioritized by three methods. The “knowledge-based” approach involved expression analysis and inclusion of existing annotations and term extension by CANDID or STRING network-based analysis, ultimately leading to the output of a gene list. Probes inside and within regulatory regions of these genes were then selected for association analysis. Probe results for the STRING arm are shown as an example (8,801 SNPs representing approximately 815 prioritized genes). In parallel, SNPs were prioritized by Random Forest analysis, which recursively partitioned the data to reveal SNPs with the highest likelihood of successful association results. Finally, fitSNPs (derived independently of this study) represented on the CNV370 array were analyzed for association.

FIG. 3 is a table listing SNPs identified as being associated with BAV appearing two or more times amongst the three categories (C=CANDID; S=STRING; F=fitSNP). Black squares indicate that the SNP had an uncorrected p-value within the lowest 100 uncorrected p-values generated in the study; grey squares indicate that the SNP was not within the lowest 100 uncorrected p-values generated in the study. The remaining column headings are gene symbol, Ensembl-51 consequence type, odds ratio (OR), and unadjusted p-value.

FIG. 4 is a schematic showing the BAV-associated haplotype spanning PDIA2 and AXIN1. Data from three approaches and relevant genomic features extracted from the Ensembl 54 database are depicted. At the top of the plot is an ideogram depicting a location on chromosome 16. The small box on the left-hand side of the ideogram delimits a region between base pair 212416 and 407490, which is displayed immediately below the ideogram and labeled “bp,” which also indicates the 5′ to 3′ orientation of the plot. Annotated gene content is displayed on a positive (denoted by “+”) and negative (denoted by “−”) strand. The four graphical data panes indicate the relative recombination rate in centimorgans per megabase (RR cM/Mb) as derived from HapMap build 36. The STRING-log(p), CANDID-log(p), and fitSNPs-log(p) data panes depict the -log10 uncorrected p-values observed in each of the three indicated schemes. All probes analyzed in the region by each respective schema are represented by a peak. The region between the two tallest peaks in the STRING and CANDID plots delineates the observed haplotype detailed in FIG. 5.

FIG. 5 is a haplotype analysis for PDIA2 and AXIN1. “|” indicates that the haplotype is grouped with the haplotype above it. Numbers in parentheses represent a 95% confidence interval. (Odds ratio affected=OR (A); odds ratio unaffected=OR (U); frequency affected=Freq A; frequency unaffected=Freq U; frequency population (control and experimental)=Freq P; peptide shift=PS. Haplotype bases in italics signify the presence of a peptide-shifting variant.)

FIG. 6 is a haplotype analysis for ENG. “|” indicates that the haplotype is grouped with the haplotype above it. Numbers in parentheses represent a 95% confidence interval. (Odds ratio affected=OR (A); odds ratio unaffected=OR (U); frequency affected=Freq A; frequency unaffected=Freq U; frequency population (control and experimental)=Freq P; peptide shift=PS. Haplotype bases in italics signify the presence of a peptide-shifting variant.)

FIG. 7 is a haplotype analysis for BAT2/3. “|” indicates that the haplotype is grouped with the haplotype above it. Numbers in parentheses represent a 95% confidence interval. (Odds ratio affected=OR (A); odds ratio unaffected=OR (U); frequency affected=Freq A; frequency unaffected=Freq U; frequency population (control and experimental)=Freq P; peptide shift=PS. Haplotype bases in italics signify the presence of a peptide-shifting variant.) The likelihood ratio for the associated haplotype has a chi-square value of 15.1 and a p-value of 0.0351.

FIG. 8 is a haplotype analysis for ZNF385D. “|” indicates that the haplotype is grouped with the haplotype above it. Numbers in parentheses represent a 95% confidence interval. (Odds ratio affected=OR (A); odds ratio unaffected=OR (U); frequency affected=Freq A; frequency unaffected=Freq U; frequency population (control and experimental)=Freq P; peptide shift=PS. Haplotype bases in italics signify the presence of a peptide-shifting variant.)

FIG. 9 is a table showing the lowest observed significant corrected p-values of the top SNPs selected by the four gene/SNP selection strategies by prioritization category. Values observed at SNPs are bolded (NP =not present in group).

### DETAILED DESCRIPTION

A bicuspid aortic valve (BAV) is a highly heritable congenital heart defect. The low frequency of BAV limits our ability to perform genome-wide association studies. However, using a variety of a priori SNP selection techniques, we identified haplotypes in several chromosomal regions that appear to be associated with BAV (e.g., chromosomal regions containing AXIN1-PDIA2, ENG, BAT2/3, and/or ZNF385D gene(s)) and describe herein methods of identifying a subject with a BAV through the detection of one or more BAV-associated SNPs in a sample (e.g., a sample containing a nucleic acid) obtained from the subject.

### Bicuspid Aortic Valve

The present invention features methods for identifying subjects with a bicuspid aortic valve by detecting one or more BAV-associated SNPs, described herein.

A bicuspid aortic valve is a defect of the aortic valve that results in the formation of two leaflets or cusps instead of the normal three. The bicuspid aortic valve may not be completely effective at stopping blood from leaking back into the heart (i.e., aortic regurgitation) and may become stiff and not open and close properly (i.e., aortic stenosis). In addition, a subject with BAV may have an enlarged aorta. Although often asymptomatic, subjects with a failing BAV may experience valve leakage, calcification of the valve, heart murmur, heart enlargement, infective endocarditis, aortic complications (e.g., dilation or dissection), fatigue, chest pain, breathing difficulty, rapid or irregular heartbeat, loss of consciousness, or pale skin. A bicuspid aortic valve is congenital and heritable.

Additional methods to diagnose a BAV that may be used in conjunction with the methods described herein include, e.g., chest x-rays, magnetic resonance imaging (MRI), electrocardiography, angiography, identifying differences in blood pressure between upper and lower extremities, assessing family history of BAV and other congenital heart defects, and identifying symptoms of a BAV.

Once a subject has been diagnosed with a BAV, treatment may include surgery to repair the valve, valve replacement surgery, cardiac catheterization, administration of inotropic agents and/or diuretics, and/or antibiotic treatment.

### Detection of Single Nucleotide Polymorphisms (SNPs)

Methods for detecting SNPs present in a polynucleotide sequence involve procedures that are well known in the art (e.g., amplification of nucleic acids). See, e.g., Single Nucleotide Polymorphisms: Methods and Protocols, Pui-Yan Kwok (ed.), Humana Press, 2003. Although many detection methods employ polymerase chain reaction (PCR) steps to detect SNPs of a polynucleotide, other amplification protocols may also be used including, e.g., ligase chain reactions, strand displacement assays, and transcription-based amplification systems.

In general, detection of SNPs or other polymorphisms can be performed using oligonucleotide primers and/or probes. Oligonucleotides can be prepared by any suitable method (e.g., chemical synthesis). Oligonucleotides can be synthesized using commercially available reagents and instruments. Alternatively, they can be purchased through commercial sources. Methods of synthesizing oligonucleotides are well known in the art (see, e.g., Narang et al., Meth Enzymol. 68: 90-99, 1979 and U.S. Pat. No. 4,458,066). In addition, modifications to such methods of oligonucleotide synthesis may be used, e.g., to impact enzyme behavior with respect to the synthesized oligonucleotides. For example, incorporation of modified phosphodiester linkages (e.g., phosphorothioate, methylphosphonates, phosphoamidate, or boranophosphate) into an oligonucleotide may be used to prevent cleavage of the oligonucleotide at a selected site.

The genotype of an individual for a BAV-associated polymorphism can be determined using many detection methods that are well known in the art including, e.g., hybridization using allele-specific oligonucleotides, primer extension, allele-specific ligation, sequencing, or electrophoretic separation techniques, e.g., single-stranded conformational polymorphism (SSCP) and heteroduplex analysis. Exemplary assays include 5′-nuclease assays, template-directed dye-terminator incorporation, molecular beacon allele-specific oligonucleotide assays, single-base extension assays, and SNP scoring by real-time pyrophosphate sequences. Analysis of amplified sequences can be performed using various technologies such as microarrays, fluorescence polarization assays, and matrix-assisted laser desorption ionization (MALDI) mass spectrometry.

Detecting the presence of a SNP is generally performed by analyzing a sample (e.g., a biological sample containing nucleic acid) that is obtained from an individual. Often, the biological sample includes genomic DNA. The genomic DNA is typically obtained from blood samples, but may also be obtained from other cells (e.g., cardiomyocytes or lymphocytes) or tissues (e.g., cardiac tissue). For example, the biological sample may include cells, protein or membrane extracts of cells, or blood, or biological fluids. Biological samples may be obtained by standard methods including, e.g., venous puncture and surgical biopsy.

It is also possible to analyze RNA samples for the presence of polymorphic alleles. For example, mRNA can be used to determine the genotype of an individual at one or more BAV-associated polymorphic sites. In this case, the biological sample is obtained from cells in which the target nucleic acid is expressed, e.g., cardiomyocytes. Such an analysis can be performed by first reverse-transcribing the target RNA using, for example, a viral reverse transcriptase, and then amplifying the resulting cDNA or, alternatively, using a combined high-temperature reverse-transcription-polymerase chain reaction (RT-PCR), as described in U.S. Pat. Nos. 5,310,652; 5,322,770; 5,561,058; 5,641,864; and 5,693,517.

Other nucleic acid samples that may be analyzed include, e.g., genomic fragmented DNA, PCR-amplified DNA, and cDNA.

Frequently used methodologies for the analysis of biological samples to detect SNPs are briefly described. However, any method known in the art can be used in the invention to detect the presence of SNPs.

Allele-Specific Hybridization

This technique, also referred to as allele-specific oligonucleotide (ASO) hybridization, relies on distinguishing between two DNA molecules differing by one base by hybridizing an oligonucleotide probe that is specific for one of the variants to an amplified product obtained from amplifying the nucleic acid obtained from the biological sample. This method typically employs short oligonucleotides, e.g., oligonucleotides 15-20 bases in length. The oligonucleotide probes are designed to hybridize to one variant, but not to another variant. Hybridization conditions should be sufficiently stringent so that there is a significant difference in hybridization intensity between alleles, whereby an oligonucleotide probe hybridizes to only one of the alleles. The amount and/or presence of an allele may be determined by measuring the amount of allele-specific oligonucleotide that is hybridized to the sample. Typically, the oligonucleotide is labeled (e.g., with a fluorescent label). For example, an allele-specific oligonucleotide may be applied to immobilized oligonucleotides representing BAV-associated SNP sequences. After stringent hybridization and subsequent washing, fluorescence intensity is measured for each SNP oligonucleotide.

According to the invention, SNPs can be identified in a high throughput fashion via a microarray that allows the identification of one or more SNPs at any given time. Such microarrays are described, for example, in WO 00/18960. An array usually involves a solid support on which nucleic acid probes have been immobilized. These arrays may be produced using mechanical synthesis methods or light-directed synthesis methods that incorporate a combination of photolithographic methods and solid-phase synthesis methods. (See, for example, Fodor et al., Science 251: 767-777, 1991, and U.S. Pat. Nos. 5,143,854 and 5,424,186, each of which is hereby incorporated by reference.) Although a planar array surface is typically used, the array may be fabricated on a surface of virtually any shape or even a multiplicity of surfaces. Arrays may be nucleic acids on beads, beadchips (e.g., Illumina's 370CNV Infinium chemistry-based whole genome DNA analysis beadchip), fibers (e.g., fiber optics), glass, or any other appropriate substrate.

In one example, SNP arrays utilize ASO hybridization to detect polymorphisms. SNP arrays include immobilized nucleic acid sequences or target sequence, one or more labeled allele-specific oligonucleotide probes, and a detection system that records and interprets the hybridization signal. To achieve relative concentration independence and minimal cross-hybridization, raw sequences and SNPs of multiple databases are scanned to design the probes. Each SNP on the array is interrogated with different probes.

Other suitable assay formats for detecting hybrids formed between probes and target nucleic acid sequences in a sample are known in the art and include the immobilized target (e.g., dot-blot) formats and immobilized probe (e.g., reverse dot-blot or line-blot) assay formats. Dot-blot and reverse dot-blot assay formats are described in U.S. Pat. Nos. 5,310,893; 5,451,512; 5,468,613; and 5,604,099, each incorporated herein by reference.

Allele-Specific Primers

Polymorphisms are also commonly detected using allele-specific amplification or primer extension methods. These reactions typically involve use of primers that are designed to specifically target a polymorphism via a mismatch at the 3′-end of a primer. The presence of a mismatch affects the ability of a polymerase to extend a primer when the polymerase lacks error-correcting activity. For example, to detect an allele sequence using an allele-specific amplification- or extension-based method, a primer complementary to one allele of a polymorphism is designed such that the 3′-terminal nucleotide hybridizes at the polymorphic position. The presence of the particular allele can be determined by the ability of the primer to initiate extension. If the 3′-terminus is mismatched, the extension is impeded.

In some embodiments, the primer is used in conjunction with a second primer in an amplification reaction. The second primer hybridizes at a site unrelated to the polymorphic position. Amplification proceeds from the two primers leading to a detectable product, signifying the particular allelic form is present. Allele-specific amplification- or extension-based methods are described, for example, in WO 93/22456 and in U.S. Pat. Nos. 5,137,806; 5,595,890; 5,639,611; and 4,851,331.

Detectable Probes

Genotyping can also be performed using a TaqMan (Applied Biosystems) (or 5′-nuclease) assay, as described in U.S. Pat. Nos. 5,210,015; 5,487,972; 5,491,063; 5,571,673; and 5,804,375.

The TaqMan probe principle relies on the 5′→3′ nuclease activity of Taq polymerase to cleave a dual-labeled probe during hybridization to the complementary target sequence and fluorophore-based detection. TaqMan probes consist of a fluorophore covalently attached to the 5′-end of the oligonucleotide probe and a quencher at the 3′-end. Several different fluorophores (e.g., 6-carboxyfluoresccin or tetrachlorofluorescin) and quenchers (e.g., tetramethylrhodamine or dihydrocyclopyrroloindole tripeptide) may be used. The quencher molecule quenches the fluorescence emitted by the fluorophore when excited by the cycler's light source via fluorescence resonance energy transfer. As long as the fluorophore and the quencher are in proximity, quenching inhibits a fluorescence signal.

TaqMan probes are designed such that they anneal within a DNA region amplified by a specific set of primers. As the Taq polymerase extends the primer and synthesizes the nascent strand, the 5′→3′ exonuclease activity of the polymerase degrades the probe that has annealed to the template. Degradation of the probe releases the fluorophore from the probe such that the fluorophore and quencher are no longer in close proximity, thus relieving the quenching effect and allowing fluorescence of the fluorophore. Hence, fluorescence detected in, for example, a real-time PCR thermal cycler is directly proportional to the fluorophore released and the amount of DNA template present in the PCR.

The hybridization probe can be an allele-specific probe that discriminates between the SNP alleles. Alternatively, the method can be performed using an allele-specific primer and a labeled probe that binds to amplified product.

Probes detectable upon a secondary structural change are also suitable for detection of a polymorphism, including SNPs. Exemplary secondary structure or stem-loop structure probes include molecular beacons (e.g., Scorpion® primers and probes). Molecular beacon probes are single-stranded oligonucleotide probes that can form a hairpin structure in which a fluorophore and a quencher are usually placed on the opposite ends of the oligonucleotide. At either end of the probe, short complementary sequences allow for the formation of an intramolecular stem, which enables the fluorophore and quencher to come into close proximity. The loop portion of the molecular beacon is complementary to a target nucleic acid of interest. Binding of the probe to its target nucleic acid of interest forms a hybrid that results in the opening of the stem loop and a conformational change that moves the fluorophore and the quencher away from each other, leading to a more intense fluorescent signal.

DNA Sequencing and Single Base Extensions

SNPs can also be detected by direct sequencing. Methods include, e.g., dideoxy sequencing, Maxam-Gilbert sequencing, chain-termination sequencing (e.g., Sanger method), pyrosequencing, Solexa sequencing, SOLiD sequencing, or any other sequencing method known to one of skill in the art.

Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Polymorphisms may also be detected using capillary electrophoresis. Capillary electrophoresis allows identification of repeats in a particular allele. The application of capillary electrophoresis to the analysis of DNA polymorphisms is well known in the art.

Single-Strand Conformation Polymorphism Analysis

Alleles of target sequences can be differentiated using single-strand conformation polymorphism analysis, which identifies base differences by alteration in electrophoretic migration of single-stranded PCR products. Amplified PCR products can be generated as described above and heated or otherwise denatured to form single-stranded amplification products. Single-stranded nucleic acids may refold or form secondary structures, which are partially dependent on the base sequence. The different electrophoretic mobilities of single-stranded amplification products can be related to base-sequence differences between alleles of target sequences.

SNP detection methods often employ labeled oligonucleotides. Oligonucleotides can be labeled by incorporating into or onto an oligonucleotide a label detectable by spectroscopic, photochemical, biochemical, immunochemical, or chemical means. Useful labels include fluorescent dyes (e.g., fluorescein, rhodamine, Oregon green, eosin, cyanine derivatives, naphthalene derivatives, coumarin derivatives, oxadiazole derivatives, BODIPY, pyrene derivatives, proflavin, acridine orange, crystal violet, malachite green, Alexa Fluor, porphin, phtalocyanine, bilirubin, DAPI, Hoechst 33258, Lucifer yellow, or quinine), radioactive labels (e.g., ³²P), electron-dense reagents, enzymes (e.g., peroxidase or alkaline phosphatase), biotin, fluorescent proteins (e.g., green fluorescent proteins), or haptens and proteins for which antisera or monoclonal antibodies are available. Labeling techniques are well known in the art.

### SNP Detection Kits

Detection reagents can be developed and used to assay SNPs of the present invention (individually or in combination), and such detection reagents can be readily incorporated into a kit format. Accordingly, the present invention further provides SNP detection kits, including but not limited to, packaged probe and primer sets (e.g., TaqMan probe and primer sets), arrays and/or microarrays of nucleic acid molecules, and beads that contain one or more probes, primers, or other detection reagents for detecting one or more SNPs of the present invention. The kits can optionally include various electronic hardware components, containers, and devices.

### BAV Diagnosis and Screening

An association or correlation between a genotype and phenotype (e.g., bicuspid aortic valve formation) can be exploited in several ways. For example, in the case of a statistically significant association between one or more SNPs with predisposition to a disease or condition for which treatment is available, detection of such a genotype pattern in a subject may justify immediate administration of treatment or regular monitoring of the subject.

The SNPs of the invention may contribute to a BAV in a subject in different ways. Some polymorphisms may occur within a coding sequence and may contribute to the BAV phenotype by affecting protein structure. Other polymorphisms may occur in non-coding regions, but may exert phenotypic effects indirectly, e.g., by affecting replication, transcription, and/or translation. A single SNP may affect more than one phenotypic trait. Likewise, a single phenotypic trait may be affected by multiple SNPs in different genes.

The methods described herein may be used to identify a subject with BAV. Such methods include, but are not limited to, any of the following: detection of BAV that a subject may presently have; predisposition screening (i.e., determining the increased risk for a subject in developing BAV-related symptoms in the future or determining whether an individual has a decreased risk of developing BAV-related symptoms in the future); determining a particular type or subclass of BAV in a subject known to have BAV; confirming or reinforcing a previously made diagnosis of BAV; following the success of a therapeutic regimen; or pharmacogenomic evaluation of a subject to determine which therapeutic strategy that subject is most likely to respond to or to predict whether a subject is likely to respond to a particular treatment. Such diagnostic uses may be based on the presence or absence of one or more BAV-associated SNPs (e.g., SNPs present in chromosomal regions containing the AXIN1-PDIA2, ENG, BAT2/3, or ZNF385D gene(s)).

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in linkage equilibrium. In contrast, LD refers to any non-random genetic association between allele(s) at two or more different SNP sites, which is generally due to the physical proximity of the two loci along a chromosome. LD can occur when two or more SNP sites are in close physical proximity to each other on a given chromosome and, therefore, alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For diagnostic purposes, if a particular SNP site is found to be useful for identifying a subject with a BAV, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and the degree of physical separation between two or more SNP sites necessary for LD to occur can differ between different regions of the genome.

For diagnostic applications, polymorphisms that are not the actual disease- or condition-causing (i.e., causative) polymorphisms, but are in LD with such causative polymorphisms, are also useful. In such instances, the genotype of the polymorphism(s) that are in LD with the causative polymorphism are predictive of the genotype of the causative polymorphism and, consequently, predictive of the phenotype (e.g., a BAV phenotype) that is influenced by the causative SNP(s). Thus, polymorphic markers that are in LD with causative polymorphisms are useful as diagnostic markers and are particularly useful when the actual causative polymorphism(s) are unknown.

As described herein, diagnostics may be based on a single SNP or a group of SNPs that are associated with the BAV phenotype. Combined detection of a plurality of SNPs (e.g., 2, 3, 4, 5, 6, 7, 8, 9, 10, 11, 12, 13, 14, 15, 16, 17, 18, 19, 20, 25, 30, 35, 40, 45, 50, 75, 100, or more) typically increases the probability of an accurate diagnosis. To increase the accuracy of diagnosis or predisposition screening, analysis of the SNPs of the present invention can be combined with other diagnostic methods including, e.g., echocardiography, cardiac catheterization, cardiac magnetic resonance imaging, assessing family history of BAV, and identifying symptoms of a BAV.

### EXAMPLES

The present invention is illustrated by the following examples, which are in no way intended to be limiting of the invention.

### Example 1

### Application of Gene Network Analysis Techniques to Identify SNPs Associated with Bicuspid Aortic Valves

The Tufts Medical Center/Tufts University Institutional Review Board approved all studies. Study procedures were in accordance with the principles expressed in the Declaration of Helsinki.

We collected DNA from 66 probands found to have a bicuspid aortic valve, wherein the BAV was diagnosed by direct visualization at the time of aortic valve replacement or by echocardiography. Collection of DNA from subjects with a BAV (and some of their family members) began in February 2006. Blood was collected in PaxGene DNA tubes (Qiagen), and the DNA was purified using a dedicated purification kit. The concentration of DNA in each sample was measured using a PicoGreen assay (Invitrogen).

The average age of the participants at the time of study entry was 53 years, though the ages of participants in the study ranged from 18 to 85 years old. A majority of subjects were men (54 men out of 66 total participants). Among the 66 subjects with a BAV, we included in our cohort six subjects with a coarctation of the aorta (all repaired) and 19 subjects having an aneurysm of the ascending aorta (17 repaired) since the BAV-associated aortic phenotypes are considered to be independent manifestations of a single underlying gene defect with incomplete penetrance (Loscalzo et al., Am J Med Genet A 143: 1960-1967, 2007). Echocardiography demonstrated normal valve function of the BAV in 18 subjects. Moderate to severe aortic valve stenosis was detected in 38 subjects, and three subjects had isolated severe aortic valve regurgitation.

Genomic DNA was genotyped on an Illumina 370CNV array by deCode (Reykjavik, Iceland). Genotypes for 823 control individuals (average age of 43, ages ranged from 30 to 88 years old) were obtained from Illumina iControlDB (http://www.illumina.com/science/icontroldb.ilmn; accessed Jan. 4, 2010) and joined seven BAV-negative familial controls present in our dataset for a total of 830 individuals in the control cohort. Using the software package Structure and the standard methodology described by Falush et al. (Mol Ecol Notes 7: 574-578, 2007), we employed 25,000 independent simulations to model the number of potential sub-populations (K) present in our case/control cohort. The smallest value of K that appeared to describe the cohort was three. Values of K from zero to eight were analyzed for their consistency. These analyses returned no evidence of significant allele frequency divergence at any value of K within the control and experimental groups when considered singly or as a whole.

Association and locus analyses were carried out using plink v1.06 (Purcell et al., Am J Hum Genet. 81: 559-575, 2007). Baseline settings were employed for inclusion of probes and quality control (genotyping rate >80%, pruning of probes based on missingness (GENO>1) and low frequency (MAF<0), as well as the removal of heterozygous haploid genotypes (4,545 found and removed from analysis in this dataset)). The total genotyping rate across all individuals (66 cases, 830 controls) was 0.98, and the initial probe number (from which all described priority and genome-wide association study (GWAS) probes were drawn) was 311,399 after all pruning. Primary analysis of all priority sets was conducted using the same set of covariates and phenotype definitions for each set. Full plink settings for each run were “-logistic-adjust-qq-plot-sex” with the requisite additional commands for selecting input and output files. Logistic analysis was indicated for the discrete disease trait (BAV versus no BAV), and the covariate adjustment was included for gender and for returned p-values relative to number of tests performed. Lack of deep phenotype information for control population limited the ability to create more extensive covariate controls in this population.

After excluding the copy number probes (which are not represented in the Illumina-supplied control population), the dataset included 311,399 probes. The prevalence of BAV in the general population would suggest that only ˜1% of the control cohort would have a BAV. There was no evidence of significant population structure in the combined control/experimental group (Falush et al., Mol Ecol Notes 7: 574-578, 2007).

Using the Genetic Power Calculator for discrete traits (Purcell et al., Bioinformatics 19: 149-150, 2003), we determined that, with our cohort dimensions and a 1% population prevalence of BAV, power to detect at a significant association in our cohort would reach 80% at observed odds ratios greater than 2. Odds ratios of 4 or greater reach similar power with 53 individuals. By comparison, the various BAV sub-phenotypes present in our cohort each represents fewer than 18 individuals, far fewer than are required to reach any reasonable power threshold. These sub-phenotypes were therefore excluded from further analyses.

Using Cytoscape's Agilent Literature Search plug-in (Shannon et al., Genome Res. 13: 2498-2504, 2003), we compiled a list of twenty broadly topical medical subject heading (MeSH) keywords (including OMIM headings) to describe each of the major categories and outcomes of BAV and BAV-associated syndromes. Additionally, we added various gene-specific descriptors (e.g., NOTCH) to ensure full coverage of previously described BAV genetic profiles. We also included information garnered from microsatellite analysis of BAV individuals, which had identified several discrete regions of the genome thought to be involved in the development of BAV (Martin et al., Hum Genet. 121: 275-284, 2007). These core terms produced a basic protein interaction network consisting of 124 proteins (and genomic locations).

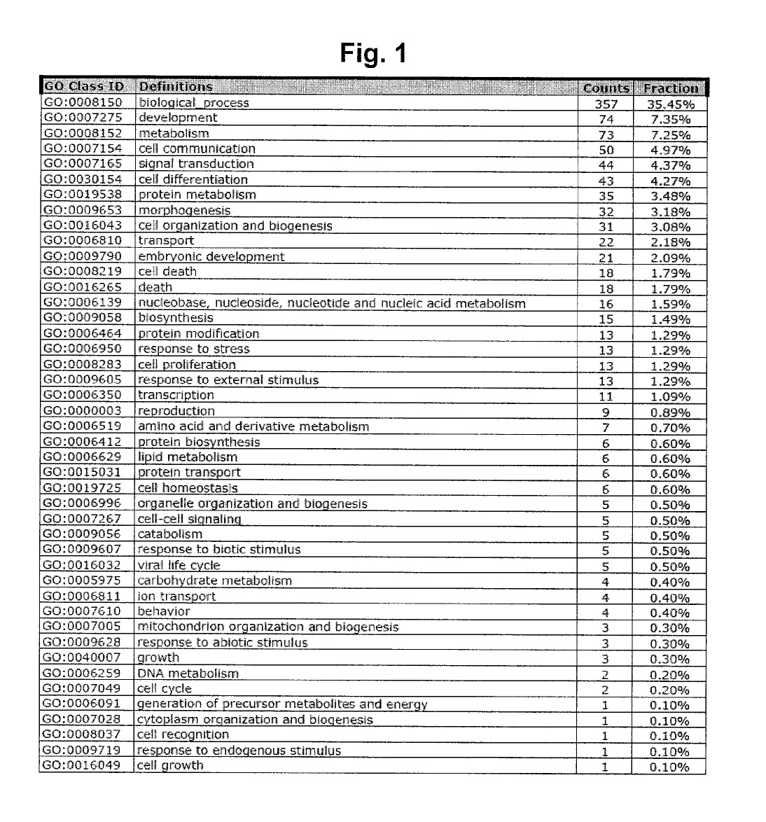

Next, we identified genes differentially expressed in the aorta of subjects with BAV compared with a subject with a normal tricuspid aortic valve (TAV) from the Gene Expression Omnibus (GEO) dataset GDS2922 (Majumdar et al., Cardiovasc Pathol. 16: 144-150, 2007). Analysis of these arrays was carried out by both parametric (limma) and non-parametric (RankProd) methods. (See, e.g., Smyth, “Linear models and empirical bayes methods for assessing differential expression in microarray experiments,” in Statistical Applications in Genetics and Molecular Biology 3: 1-26, 2004, and Hong et al., “Rank Product method for identifying differentially expressed genes with application in meta-analysis,” available at http://www.bioconductor.org/packages/2.2/bioc/html/RankProd.html.) These two approaches predominantly selected different gene-sets relative to observed expression levels in experimental (BAV, thoracic aortic aneurysm (TAA)) versus control (TAV, TAA) patients. Combining the output from the parametric and non-parametric array analyses resulted in a collection of 1,552 differentially expressed genes (limma, n=903; RankProd, n=649) that broadly mirrored previous findings from the datasets in question, but were necessarily more inclusive. Gene ontology analysis showed a broad representation from across the functional spectrum with specific ontologic classes, such as coagulation and inflammatory response, metabolism, development, and cell communication (FIG. 1). We also included the most significantly altered genes (n=41) detected in the peripheral blood of patients with TAA compared with normal individuals (GSE9106) (Wang et al., PLoS ONE 2: E1050, 2007).

Finally, we included chromosomal loci linked with BAV by microsatellite-based study (Martin et al., Human Genet. 121: 275-284, 2007, and Ellison et al., J Surg Res. 142: 28-31, 2007). Through our primary analysis of microarray data and the selection of all published and genomic loci, we constructed a knowledge-base for BAV to serve as a basis for the construction of an overall BAV-related gene network.

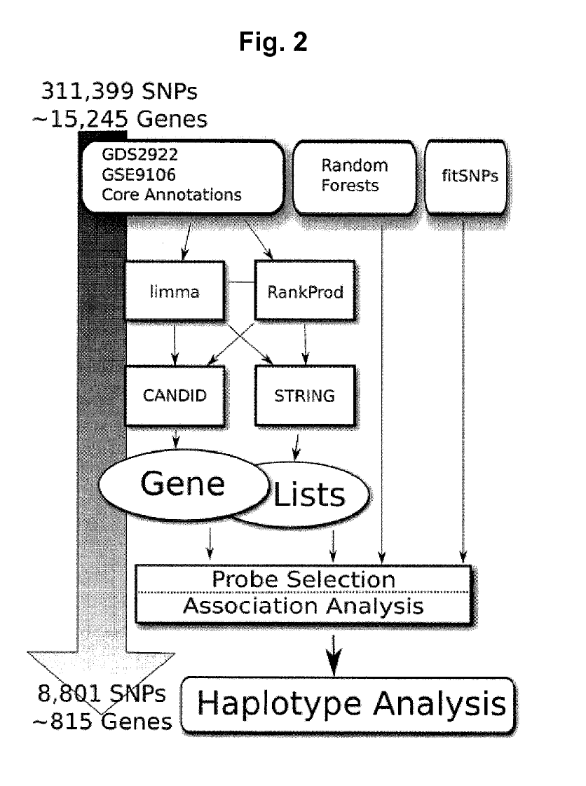

We combined the multiple sources of biological knowledge, including the RankProd and limma analyses, to create parallel interaction networks relevant to BAV using STRING (von Mering et al., Nucleic Acids Res. 35: D358-362, 2007) and CANDID (Hutz et al., Genet Epidemiol. 32: 779-790, 2008) (FIG. 2). The purpose of using dual approaches was to capture both broadly modified genes between classes as well as those genes that vary within classes while maintaining an overall differential expression level between the two groups. With regard to STRING, networks were formed with a maximum of 4 additional inter-member nodes, a “medium” confidence score of 0.4, no more than 50 interactors shown, an edge scaling of 80%, and all Active Prediction Methods selected. CANDID allows for more nuanced sub-scoring of various components. All components were weighted equally, with the exception of “Conservation,” which was given the lesser weight of 2, relative to the rest of the entries. Tissue codes 44 (heart) and 53 (cardiac myocytes) were employed. In all cases, output of proteins and their interactors were converted into genomic regions of origin by employing the UCSC Genome Table Browser and Ensembl Gene codes (ENSG) to capture various potential transcripts that may arise from a single “gene.” Additionally, a buffer region of 5 kb was added to the beginning and end of each expression region to allow for upstream or downstream control elements potentially present. These regions in hand, the intersection of CNV370 probes falling within the various regions were calculated. The number of SNPs corresponding to each class of prioritization with relative overlap between classes is shown in Table 1.

TABLE 1
Prioritized probe distribution by category

| Subclass | CANDID | STRING |
|---|---|---|
| limma only | 94 | 2,960 |
| RankProd only | 61 | 5,200 |
| limma/RankProd | 12,405 | 641 |
| Total SNPs | 12,560 | 8,801 |
| Random Forests | 222 | 180 |
| fitSNPs | 554 | 543 |
| STRING/CANDID | 1,397 | 1,397 |

STRING took the outputs of the limma and RankProd expression analyses and created substantially different output networks, reflecting the nature of the input, with RankProd ultimately generating a gene list composed of 5,841 prioritized SNPs, and limma generating a gene list composed of 3,601 SNPs. An additional 641 probes were shared between methods. CANDID, alternatively, produced highly similar lists from the same limma and RankProd inputs, ultimately differing at only 155 out of 13,769 selected SNPs.

Separately, we identified the 8,801 Functionally Interpolating SNPs (fitSNPs) present on the Illumina 370CNV array. FitSNPs were derived through extensive analysis of public array datasets (Chen et al., Genome Biology 9: R170, 2008). While not selected relative to specific disease or expression outcome, fitSNPs represent a set of markers enriched for functionally relevant variants (e.g., non-synonymous coding region polymorphisms) from across the genome. From the 370CNV array, 1,532 probes were selected both by CANDID (12% of all CANDID SNPs) and by STRING (15% of all STRING SNPs). The fitSNPs overlapped CANDID at 554 probes (4.4%) and STRING at 543 probes (6.2%). 97 SNPs were selected by these three approaches (CANDID, STRING and fitSNPs). Full comparisons between probes selected by CANDID, STRING, Random Forests, and fitSNPs are shown in Table 1 and in Table 2. Table 2 shows probe distribution by category (categories in italics represent shared probes from those respective classes).

TABLE 2
Ab initio probe distribution by category

| Subclass | FitSNPs | Random Forests |
|---|---|---|
| Random Forests | 123 | 6,322 |
| FitSNPs | 8,100 | 123 |
| Prioritized | 1,097 | 402 |

We considered genes identified by more than one strategy to have the greatest potential for a role in BAV. To test this hypothesis, we determined the association of each prioritized SNP for differentiating case (BAV) from control status. When we compared the top 100 SNPs (by p-value) from each strategy, three chromosomal regions were selected by all three strategies: AXIN1-PDIA2, ENG, and BAT2-BAT3.

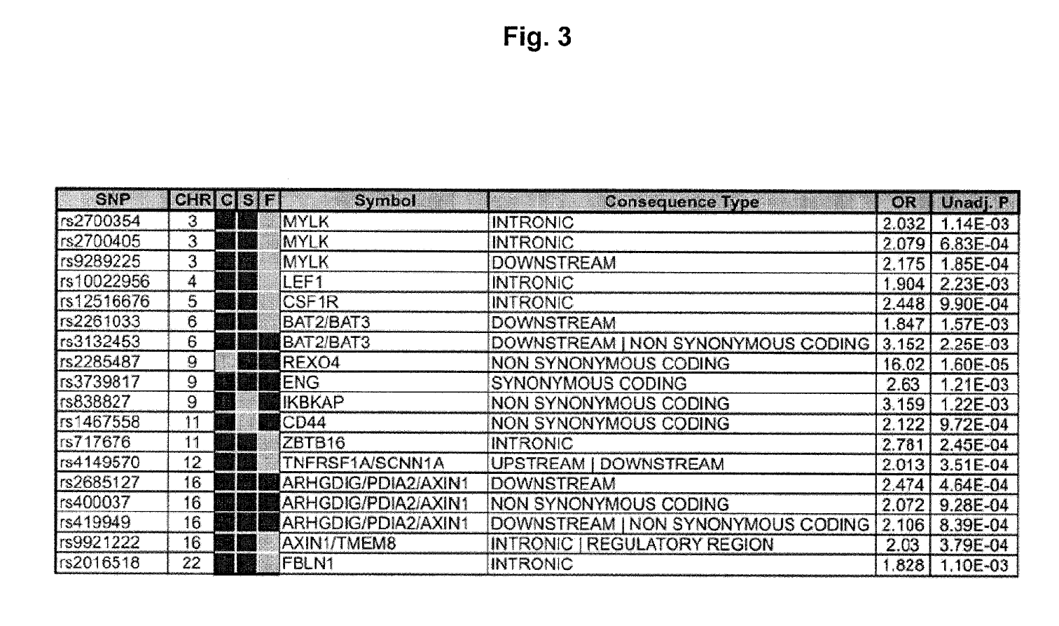

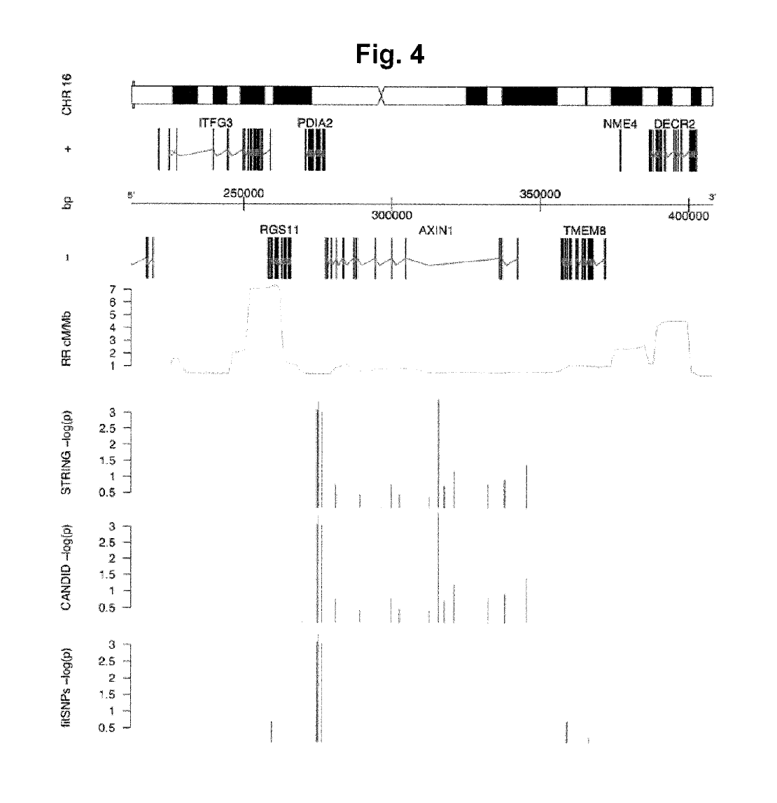

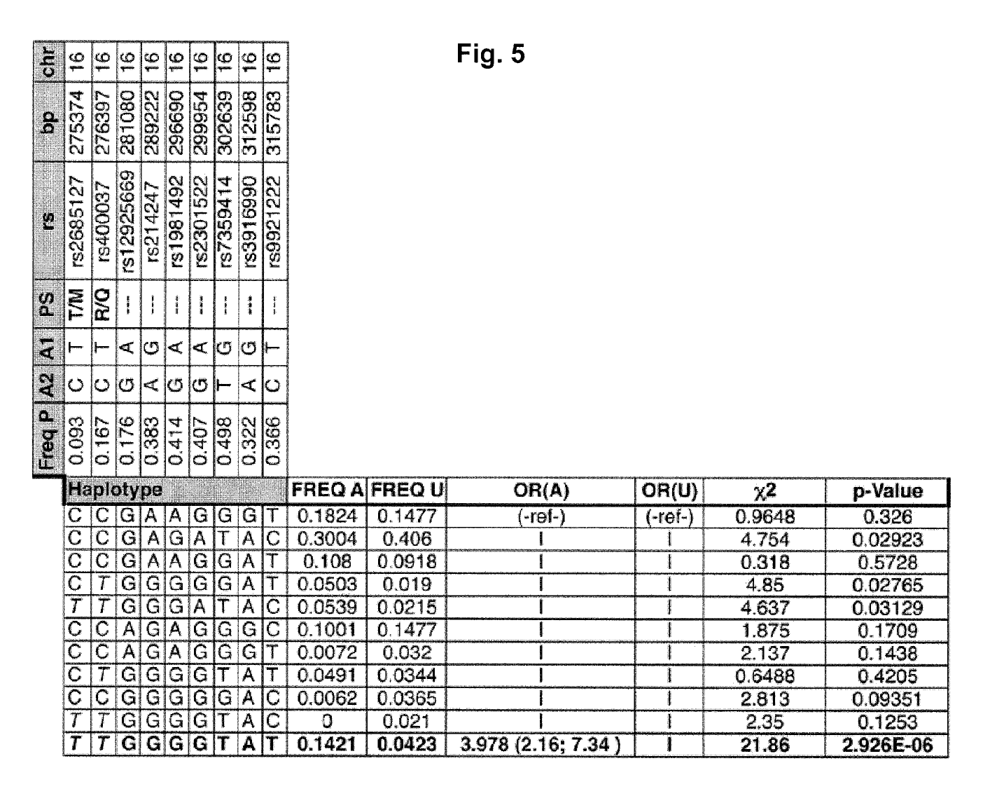

AXIN1-PDIA2 Haplotype Is Associated with BAV. STRING, CANDID, and fitSNPs identified a concentration of SNPs in chromosome 16p13.3 within a region that includes AXIN1 and PDIA2. AXIN1 was selected by CANDID and STRING because its expression in aorta from subjects with BAV compared with TAV was significantly different by limma (adjusted p-value of 3.79×10⁻⁴⁹). Four SNPs selected by all three approaches within this region (FIG. 3) showed associations with BAV that did not surpass correction for multiple testing (FIGS. 3 and 4). There was significant linkage disequilibrium (LD) observed in the locus, suggesting a haplotype block structure. Eleven haplotypes derived from nine AXIN1-PDIA2 region SNPs (FIG. 5) were identified in this region. The observed block structure in our case-control cohort recapitulates that observed by the relevant European HapMap data (The International HapMap Consortium, Nature 449: 851-861, 2007). The TTGGGGTAT haplotype showed the strongest association with BAV and surpassed the Bonferroni correction for multiple testing (p-value of 2.926×10⁻⁶, OR=3.978). Further narrowing or widening of this window reduced the association of the locus, strongly implying that variants within this regional block unit are associated to the BAV phenotype.

Haplotype analysis was unable to discriminate whether the variants in AXIN 1 or PDIA2 were driving the association with BAV in this gene-rich region. Indeed, SNPs observed within PDIA2 are in high LD with SNPs in AXIN1 regulatory regions and vice-versa. However, the three SNPs with the strongest individual association with BAV are all non-synonymous polymorphisms of PDIA2: -T286M (rs2685127), -K185E (rs419949), and -Q388R (rs400037). Of these, rs419949 lies outside the most strongly associated haplotype. These results suggest that the co-occurrence of two of these three PDIA2 protein-coding changes is associated with increased odds of BAV. However, as the bulk of the haplotype resides in AXIN1, a gene in a pathway relevant to heart valve formation (Hurlstone et al., Nature 425: 633-637, 2003), we cannot exclude a role for a primary genetic variant located in either PDIA2, AXIN1, or both as contributing to the observed association with BAV. There are currently no additional known non-synonymous coding SNPs located within this region beyond those represented in the collected genotypes.

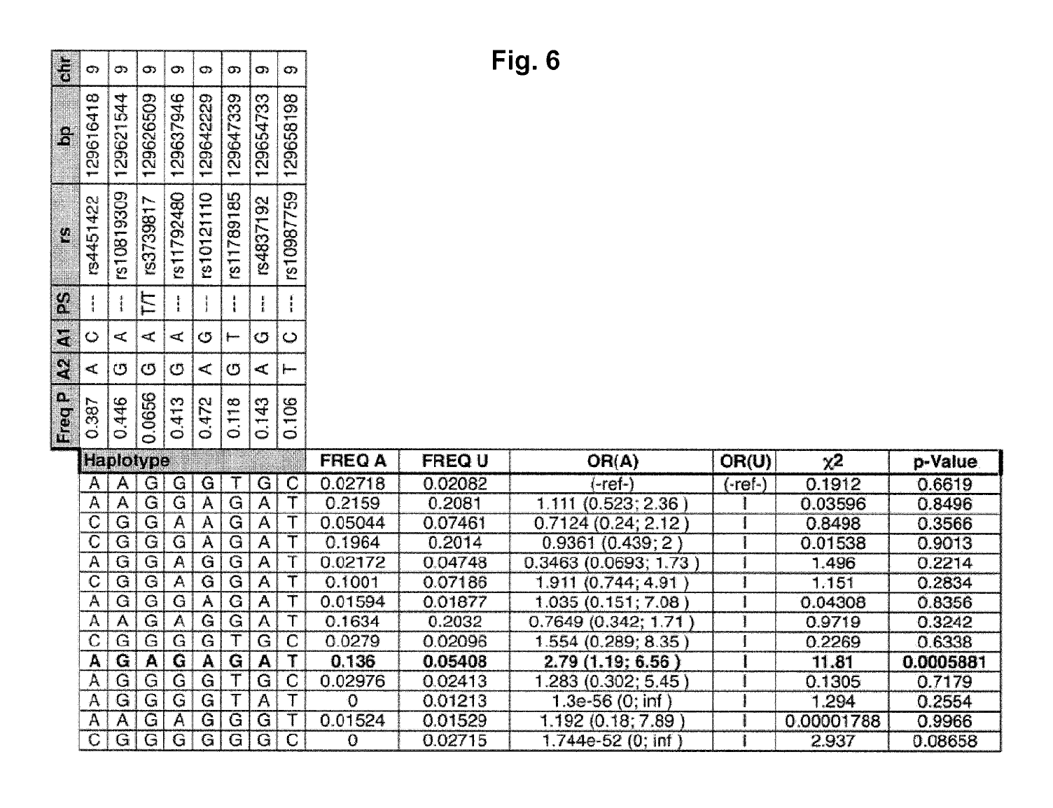

Endoglin (ENG) Haplotype Is Associated with BAV. SNPs within the endoglin gene (ENG) were prioritized for analysis by STRING, CANDID, and fitSNPs. ENG was initially selected for inclusion by RankProd analysis that found differential expression in aortic aneurismal tissue from patients with BAV compared with TAV (GDS2922). Haplotype analysis identified one block including a conservative coding region variant (ENG-T343T, rs3739817) associated with BAV (p-value of 5.88×10⁻⁴, OR=2.79) (FIG. 6). ENG-T343T (rs3739817) appears to be the critical SNP within the haplotype, as presence of the minor allele at this locus segregates with BAV across the region. Though the predicted amino acid change is synonymous (T/T), recent work suggests that even conservative alterations may yield functionally unique outcomes (Kudla et al., Science 324: 255-258, 2009). The possibility also exists for a novel, causative variation in high LD with this haplotype/SNP not present in our analysis; no known variants of this type are present within the region.

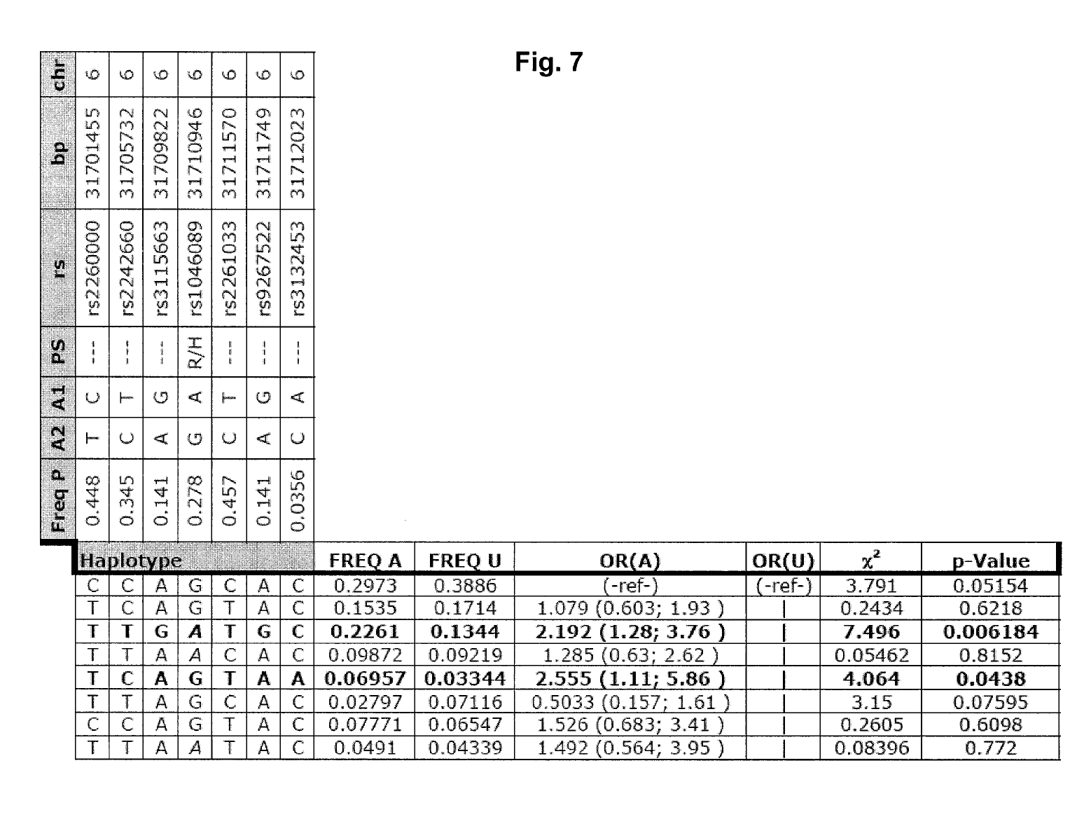

Additional Loci. The BAT2/3 locus is located at chromosome 6, and two haplotypes in this region are each weakly associated with BAV (FIG. 7). However, detailed analysis of this haplotype fails to show a single, clear association with trait. Similarly, the remaining repeated-hit loci at MYLK, LEF1, CSF1R, REXO4, CD44, ZBTB16, and FBNL1, though containing associated SNPs, do not return a single associated haplotype that spans the region of interest, typically due to low probe counts, very large regions, or low frequency of the putative associated haplotypes within our population. While these loci may be significant in BAV, further study is required to clarify the location and identity of causative variant(s) that may be within these regions.

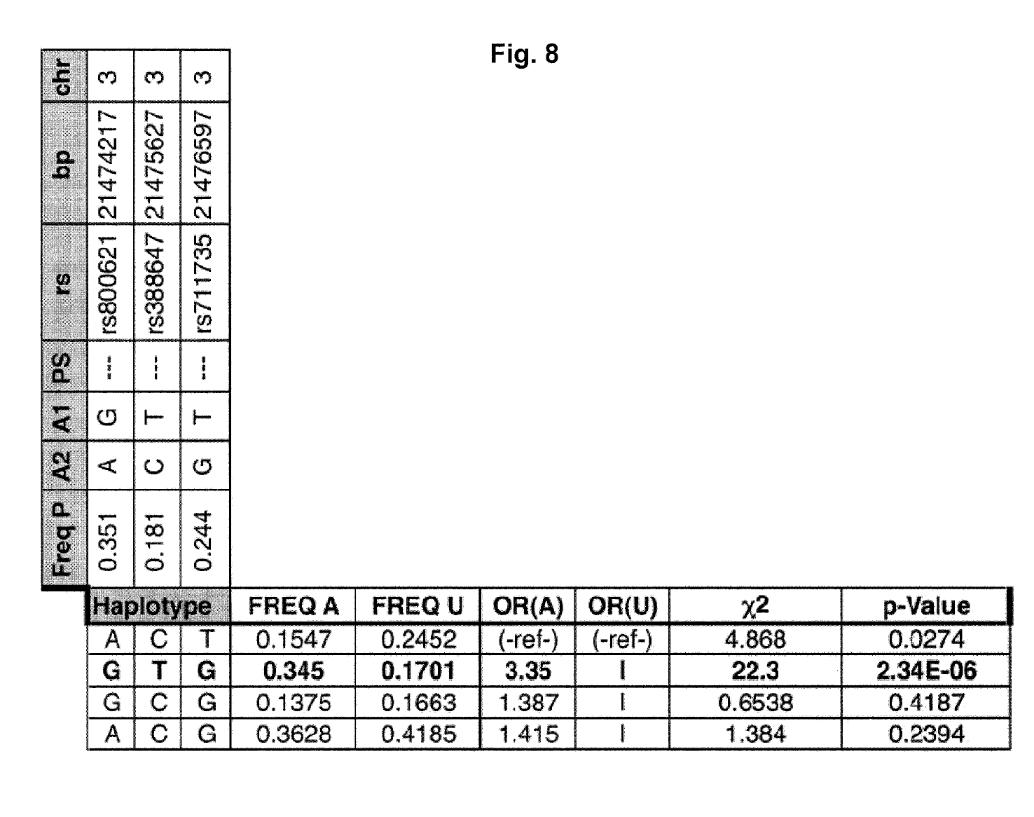

Random Forests Analysis. As a complementary strategy that is not dependent on existing knowledge, we performed a Random Forests analysis to select SNPs from our genome-wide dataset with the greatest information content. Random Forests analysis was performed using the R package random Forests (Liaw and Wiener, “Classification and regression by random Forest,” R News 2: 18-22, 2002) with the following non-default settings: 10,000 trees; 5,000 iterations; and importance=TRUE. Input data comprised all SNPs with a non-zero variance. Total input SNPs numbered 267,196, yielding 6,322 “important” SNPs for further analysis, approximately equal in dimension to that used by the supervised approaches. Fewer than five percent of the SNPs selected by Random Forests were also selected by either CANDID, STRING, or fitSNPs (Table 1). Association analysis identified one SNP associated with BAV (rs388647, OR=4.562, p-value of 0.03201 following correction for multiple testing) located on chromosome 3 within the RefSeq transcript zinc finger protein 385D (ZNF385D; NM_{—}024697.2). Low probe density in this region on the Illumina 370CNV array (there are only two other probes located within 5 kb of the associated SNP) prevents a more detailed haplotype analysis. However, the associated SNP and its two nearest neighbors form a stable haplotype that spans an exon (FIG. 8).

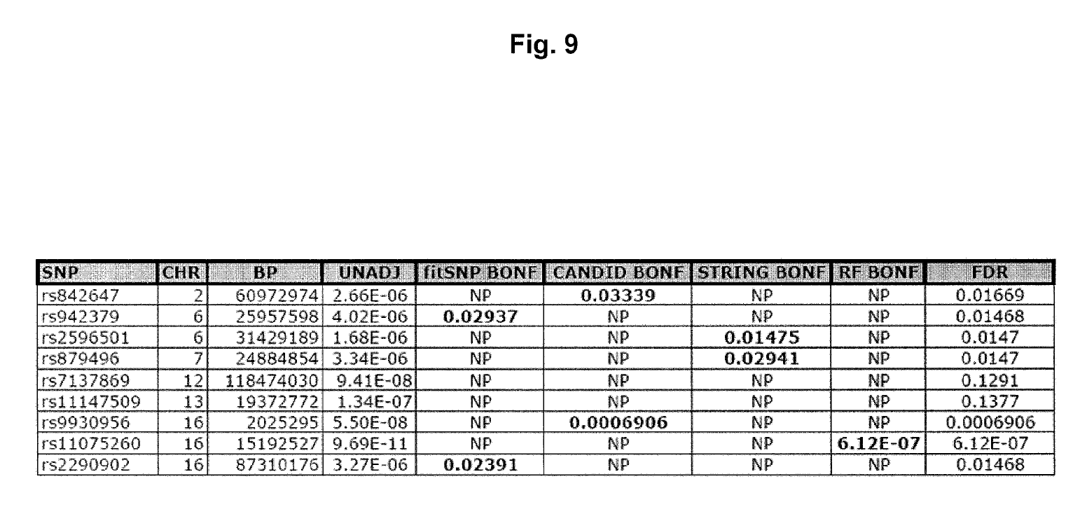

Comparison of the Four Gene/SNP Selection Strategies. We compared the top SNPs selected by the four gene/SNP selection strategies (FIG. 9). Each selection strategy picked a different top SNP. CANDID selected the SNP with the strongest BAV association (rs9930956) located in an intron of the gene SLC9A3R2 (uc002coj.2).

### Other Embodiments

From the foregoing description, it is apparent that variations and modifications may be made to the invention described herein to adopt it to various usages and conditions. Such embodiments are also within the scope of the following claims.

All patents, patent applications, and publications mentioned in this specification are herein incorporated by reference to the same extent as if each independent patent, patent application, or publication was specifically and individually indicated to be incorporated by reference.

What is claimed is:

1. A method for identifying a subject with a bicuspid aortic valve (BAV), said method comprising detecting in a biological sample obtained from said subject at least one single nucleotide polymorphism (SNP) in one or more BAV-associated chromosomal regions selected from AXIN1-PDIA2, ENG, BAT2/3, and/or ZNF385D, wherein the presence of said at least one SNP identifies said subject as having a BAV.

2. The method of claim 1, wherein said BAV-associated chromosomal region is AXIN1-PDIA2 and said at least one SNP is rs2685127, rs419949, rs12925669, rs214247, rs1981492, rs2301522, rs7359414, rs3916990, rs9921222, and/or rs400037.

3. The method of claim 1, wherein said BAV-associated chromosomal region is ENG and said at least one SNP is rs4451422, rs10819309, rs3739817, rs11792480, rs10121110, rs11789185, rs4837192, and/or rs10987759.

4. The method of claim 1, wherein said BAV-associated chromosomal region is BAT2/3 and said at least one SNP is rs2261033 and/or rs3132453.

5. The method of claim 1, wherein said BAV-associated chromosomal region is ZNF385D and said at least one SNP is rs388647, rs800621, and/or rs711735.

6. The method of claim 1 any one of claims 1 5, wherein said biological sample comprises nucleic acid.

7. The method of claim 6, wherein said nucleic acid is one or more of DNA, genomic DNA, RNA, cDNA, hnRNA, or mRNA.

8. The method of claim 6, wherein said nucleic acid is extracted and amplified.

9. The method of claim 1, wherein said biological sample is obtained from heart tissue or peripheral blood.

10. The method of claim 1, wherein said detecting step comprises one or more of oligonucleotide microarray analysis, allele-specific hybridization, allele-specific polymerase chain reaction (PCR), 5′ nuclease digestion, molecular beacon assay, oligonucleotide ligation assay, size analysis, or nucleic acid sequencing.

11. The method of claim 1, wherein said subject is human.

12. The method of claim 11, wherein said subject has a personal or family history of heart defects or heart disease.

13. A kit comprising an assay for detecting at least one SNP in one or more BAV-associated chromosomal regions selected from AXIN1-PDIA2, ENG, BAT2/3, and/or ZNF385D, wherein the presence of at least one SNP identifies said subject as having a BAV.

14. The kit of claim 13, wherein said assay comprises nucleic acid probes and/or primers specific to said at least one SNP in one or more BAV-associated chromosomal regions selected from AXIN1-PDIA2, ENG, BAT2/3, and/or ZNF385D.

15. The kit of claim 14, wherein said BAV-associated chromosomal region is AXIN1-PDIA2 and said at least one SNP is rs2685127, rs419949, rs12925669, rs214247, rs1981492, rs2301522, rs7359414, rs3916990, rs9921222, and/or rs400037.

16. The kit of claim 14, wherein said BAV-associated chromosomal region is ENG and said at least one SNP is rs4451422, rs10819309, rs3739817, rs11792480, rs10121110, rs11789185, rs4837192, and/or rs10987759.

17. The kit of claim 14, wherein said BAV-associated chromosomal region is BAT2/3 and said at least one SNP is rs2261033 and/or rs3132453.

18. The kit of claim 14, wherein said BAV-associated chromosomal region is ZNF385D and said at least one SNP is rs388647, rs800621, and/or rs711735.

19. The kit of claim 13, further comprising instructions for correlating said assay results with said presence of BAV in said subject.

20. A microarray comprising oligonucleotide probes capable of hybridizing under stringent conditions to one or more nucleic acid molecules having at least one SNP in one or more BAV-associated chromosomal regions selected from AXIN1-PDIA2, ENG, BAT2/3, and/or ZNF385D.

21. The microarray of claim 20, wherein said BAV-associated chromosomal region is AXIN1-PDIA2 and said at least one SNP is rs2685127, rs419949, rs12925669, rs214247, rs1981492, rs2301522, rs7359414, rs3916990, rs9921222, and/or rs400037.

22. The microarray of claim 20, wherein said BAV-associated chromosomal region is ENG and said at least one SNP is rs4451422, rs10819309, rs3739817, rs11792480, rs10121110, rs11789185, rs4837192, and/or rs10987759.

23. The microarray of claim 20, wherein said BAV-associated chromosomal region is BAT2/3 and said at least one SNP is rs2261033 and/or rs3132453.

24. The microarray of claim 20, wherein said BAV-associated chromosomal region is ZNF385D and said at least one SNP is rs388647, rs800621, and/or rs711735.
